(19) United States
(12) Patent Application Publication
(10) Pub. No.: US 2018/0042919 A1
(43) Pub. Date: Feb. 15, 2018

(54) PARP Inhibitors for the Treatment of Major Depressive Disorder and Related Conditions

(71) Applicant: East Tennessee State University Research Foundation, Johnson City, TN (US)

(72) Inventors: Gregory A. Ordway, Johnson City, TN (US); Attila Szebeni, Johnson City, TN (US); Russell Brown, Johnson City, TN (US)

(21) Appl. No.: 15/674,717

(22) Filed: Aug. 11, 2017

Related U.S. Application Data
(60) Provisional application No. 62/373,647, filed on Aug. 11, 2016.

(51) Int. Cl. A61K 31/4725; A61K 31/138; A61K 31/166

(52) U.S. Cl. CPC A61K 31/4725; A61K 31/166; A61K 31/138

(57) ABSTRACT

Pharmaceutical formulations comprising an inhibitor of poly(ADP)-ribose) polymerase-1 (PARP1) and their use for the treatment of major depressive disorder and conditions that share at least one of the two major defining symptoms of major depressive disorder.

### CROSS-REFERENCE TO RELATED APPLICATION

This application depends from and claims priority to U.S. Provisional Application No.: 62/373,647 filed Aug. 11, 2016, the entire contents of which are incorporated herein by reference.

### TECHNICAL FIELD

The disclosure relates to the pharmaceutical and mental health fields. Specifically, the present disclosure relates to pharmaceutical formulations comprising an inhibitor of poly(ADP)-ribose) polymerase-1 (PARP1) and their use for the treatment of major depressive disorder and conditions that share at least one of the two major defining symptoms of major depressive disorder. The presently-disclosed subject matter further relates to pharmaceutical formulations comprising a PARP1 inhibitor that are administered in conjunction with a serotonin reuptake inhibitor for the treatment of major depressive disorder and conditions that share at least one of the two major defining symptoms of major depressive disorder.

### BACKGROUND

Major depressive disorder (MDD) affects 13-14 million people annually in the U.S. Many patients suffering from depressive disorders are refractory to treatment with currently available antidepressant medications, while many more exhibit only a partial response. Highlighting this issue is the fact that antidepressants are ineffective in approximately ⅓ of patients, and this is despite trials with multiple drugs or drug combinations. Thus inadequate or incomplete treatment of MDD using currently available antidepressant drugs is a major health and economic issue. Unfortunately, antidepressants that are newer to the market have not substantially mitigated this problem because these new drugs do not demonstrate a significantly greater therapeutic efficacy than older drugs, with minor exceptions. This suggests that brain systems are disturbed in MDD that are not corrected by treatment with current antidepressants in many patients. The primary actions of currently available antidepressants modify neurotransmission along noradrenergic and serotonergic neurons. Given these therapeutic shortcomings of current antidepressants, there accordingly remains a need in the art for the identification novel drug targets to provide therapeutic alternatives to those who do not respond to existing treatments, as well as to improve the efficacy of existing antidepressants through adjuvant treatments.

### SUMMARY

Accordingly, the present disclosure relates to a novel class of antidepressant drugs. More specifically, the present disclosure relates to pharmaceutical formulations comprising an effective amount of an inhibitor of PARP1, and their use for the treatment of major depressive disorder (MDD) and conditions that share at least one of the two major defining symptoms of major depressive disorder. The inhibition of PARP1 is expected to reduce the negative impact of psychological stress on brain pathology that results from elevated PARP activity, which contributes to the development of depressive behavior. Similarly, once a patient has developed MDD, the administration of a small molecule inhibitor of PARP is expected to alleviate the symptoms of MDD.

### BRIEF DESCRIPTION OF THE DRAWINGS

FIG. 1 demonstrates data from human subjects wherein elevated levels of DNA oxidation were measured in white matter tissue from frontal cortex in major depressive disorder (MDD). Significantly elevated levels of white matter DNA oxidation were found in major depressive disorder brain donors.

FIG. 2A-B depicts the results of a study to measure levels of PARP1 gene expression in white matter in MDD. Brain white matter has a high density of two brain cell types, oligodendrocytes and astrocytes. PARP1 gene expression was markedly elevated in oligodendrocytes (FIG. 2A), and also elevated in astrocytes (FIG. 2B) in two different brain white matter regions (uncinate fasciculus and BA10) from major depressive subjects (black bars) as compared to psychiatrically normal controls (white bars).

FIG. 3 demonstrates the effects of chronic social defeat combined with chronic unpredictable stress on DNA oxidation levels in white and gray matter prefrontal cortex of rats. Chronic stress resulted in significantly elevated levels of DNA oxidation in brain white matter.

FIG. 4A-B depict the results of preliminary experiments examining the effects of 3-AB in the Porsolt swim test. 3-AB (40 mg/kg i.p.; n=8) or vehicle (n=10) was administered daily for 10 days prior to the swim test. Swim test data were collected on the 10th day of treatment, 2 h after drug or vehicle injection. Total time spent immobile in the tank (FIG. 4A) and the latency time to immobility (FIG. 4B) were measured. The asterisks indicate statistical significance (*p<0.05; **p<0.001).

FIG. 5A-B demonstrate the effect of PARP inhibitors on immobility time (FIG. 5A) and latency to immobility (FIG. 5B) in the Porsolt swim test. Rats were administered i.p. daily for 10 days prior to the swim test with either saline (vehicle: n=13), 3-AB administered at 3 different doses as noted (n=8-10 per group), or 5-AIQ (0.3 mg/kg; n=9). Additional groups of rats were administered three i.p. injections over 24 hours prior to the swim test with either fluoxetine (10 mg/kg per injection; n=10; FLX×3) or 3-AB (40 mg/kg per injection; n=7; 3-AB×3). The swim test data were collected 2 h after the final drug or vehicle injection. Asterisks indicate significant differences compared to the vehicle group (*p<0.05; **p<0.01). The results of statistical analyses of all other comparisons can be found in Table 1.

FIG. 6A-B demonstrate the effect of combined treatment of 3-AB and fluoxetine (FLX) on immobility time (FIG. 6A) and latency to immobility (FIG. 6B) in the Porsolt swim test. Rats were administered three i.p. injections over 24 hours prior to the swim test with either vehicle n=11), 3-AB (4 mg/kg; n=7), FLX (2.5 mg/kg; n=7), or 3-AB (4 mg/kg) plus FLX (2.5 mg/kg; n=6-7). The swim test data were collected 2 h after the final drug or vehicle injection. Asterisks indicate significant differences comparing each drug-treated group to the vehicle group (*p<0.01).

FIG. 7A-D depict the swim speeds (FIG. 7A and FIG. 7C) and locomotor activities (FIG. 7B and FIG. 7D) of swim test rats. FIG. 7A and FIG. 7B are data from rats of treatment groups studied in FIG. 7C and FIG. 7D are data from rats of treatment groups studied in FIG. 6A-B. Swim speed was measured during the swim test, and locomotor activity was measured 24 h after the second day of the Porsolt swim test, both of which were measured 2 h after drug or vehicle injection. There were no significant group effects observed for swim speed or locomotor activity in either experiment. Sample sizes are as noted in FIG. 5A-B and FIG. 6A-B.

FIG. 8A-B demonstrate the effect of 3-AB on sucrose preference (FIG. 8A) and interaction time (FIG. 8B) in rats exposed to repeated psychological stressors. Treatment groups included handled control rats not exposed to stressors (Control; n=7) and rats exposed to stressors and administered once daily i.p. injections of saline vehicle (Veh-Stressed; n=7), fluoxetine (10 mg/kg n=8) or 3-AB (40 mg/kg; n=7). Stressed rats were exposed to social defeat and unpredictable stress each day for 10 days. Statistical results of specific group comparisons are indicated by horizontal lines above bars, with asterisks indicating significance (*p<0.05; **p<0.01; ***p<0.0001). Statistical results of all comparisons are provided in Table 2.

FIG. 9 is a schematic diagram indicating the predicted chain of events that links psychological stress to the development of MDD. Asterisks indicate where in the pathways that inhibition of PARP activity will block psychological stress-induced compromise of the function of brains cells, thereby producing an antidepressant effect. Abbreviations are: ROS, reactive oxygen species; PARP1, poly(ADP-ribose)polymerase-1; NAD+, nicotinamide adenine dinucleotide; ATP, adenosine triphosphate.

### DETAILED DESCRIPTION

Particular details of various embodiments of the invention are set forth to illustrate certain aspects and not to limit the scope of the invention. It will be apparent to one of ordinary skill in the art that modifications and variations are possible without departing from the scope of the embodiments defined in the appended claims. More specifically, although some aspects of embodiments of the present invention may be identified herein as preferred or particularly advantageous, it is contemplated that the embodiments of the present invention are not limited to these preferred aspects.

Unless defined otherwise, all technical and scientific terms used herein have the same meaning as commonly understood by one of ordinary skill in the art to which the presently-disclosed subject matter belongs.

The presently-disclosed data is the first to demonstrate the ability of PARP inhibitors to counteract the deleterious effects of psychological stress on rodent behaviors, and to produce antidepressant-like activity. The data demonstrates that two structurally different PARP inhibitors, 3-AB and 5-AIQ, demonstrated antidepressant-like activity in the Porsolt swim test. Both 3-AB and 5-AIQ produced their antidepressant-like responses in the swim test at doses that did not significantly affect locomotor activity or swim speed, suggesting that reduced immobility produced by these drugs was not secondary to a stimulant effect of the compounds. Further, 3-AB protected rats from the development of anhedonia and deficits in social interaction following repeated exposure to psychological stressors. In addition, the combination of 3-AB and serotonin reuptake inhibitor (i.e., fluoxetine) produce antidepressant-like effects in the swim test at doses that did not produce a significant effect for either drug when administered alone, suggesting a synergistic effect. Thus, the data demonstrates the PARP1 inhibitors can be used as an adjuvant or co-therapeutic with traditional antidepressants.

Accordingly, the presently-disclosed subject matter demonstrates a new concept of anti-depressant pharmaceutical compositions and methods that can effectively treat major depressive disorder (MDD) and conditions that share at least one of the two major defining symptoms of MDD. This new concept of anti-depressant pharmaceutical compositions and methods for treating MDD and conditions that share at least one of the two major defining symptoms of MDD in a patient include administration of a PARP1 inhibitor. In certain aspects, the pharmaceutical composition comprising a PARP1 inhibitor is administered in conjunction with a serotonin reuptake inhibitor.

In some embodiments, the presently-disclosed subject matter includes a method for treating a subject suffering from MDD, the method comprising administering an effective amount of a poly(ADP)-ribose polymerase-1 (PARP1) inhibitor. In some embodiments, the presently disclosed subject matter includes a method for treating a subject suffering from a condition that shares at least one of the two major defining symptoms of MDD, the method comprising administering an effective amount of a PARP1 inhibitor. As used herein, “a condition that shares at least one of the two major defining symptoms of MDD” is a condition characterized by either (1) depressed mood most of the day, nearly every day, for a period of at least two weeks or (2) markedly diminished pleasure or interest in all or almost all activities most of the day, nearly every day, for a period of at least two weeks, said characterizations defined and assessed in accordance with the Diagnostic and Statistical Manual of Mental Disorders, Fifth Edition. However, it should be recognized that administration of an effective amount of a PARP1 inhibitor, and thus the pharmaceutical compositions and methods disclosed herein, can be used to treat a subject suffering from dysthymia, post-traumatic stress disorder, general anxiety disorder, and schizophrenia.

PARP1 is a key nuclear enzyme of the DNA base excision repair apparatus that is activated by double or single strand breaks, such as can occur secondary to oxidative attack of nucleotides by free radicals. PARP1 is a member of a sub-family of three PARPs (PARP1, PARP2, and PARP3) that covalently build PAR polymers onto many different proteins as a mechanism to regulate a variety of cellular functions. PARP1 is expressed in many mammalian brain regions, and its gene expression in the brain appears to be highest among the PARP1, PARP2 and PARP3 enzymes. PAR polymers are bulky and charged and addition of PAR polymers to nuclear proteins by PARP1 can modify protein-protein and protein-DNA interactions. Target proteins of PARP1-mediated PARylation include itself, histones and transcription factors resulting in chromatin remodeling and regulation of gene transcription. DNA damage repair is facilitated by PARP1, but PARP1 also facilitates NF-κB-mediated inflammatory responses and PARP1 can directly bind to promoter regions as a transcription factor. Under conditions of excessive PARP1 activation, cell death can ensue due to depletion of cellular NAD+, the substrate of PARylation. Recent studies demonstrate that PARP1 also has PARylation-independent effects on gene expression of inflammatory mediators, and it can be activated by TNFα independently from DNA damage.

As used herein, the term “treating” relates to any treatment of a MDD or a condition that shares at least one of the two major defining symptoms of MDD, including but not limited to prophylactic treatment and therapeutic treatment. “Treating” includes any effect, e.g., lessening, reducing, modulating, or eliminating, that results in the improvement of the major depressive disorder or a condition that shares at least one of the two major defining symptoms of MDD. “Treating” or “treatment” of MDD or a condition that shares at least one of the two major defining symptoms of MDD includes: inhibiting these diseases, i.e., arresting the development of these diseases or their clinical symptoms; or relieving the these diseases or their clinical symptoms, i.e., causing temporary or permanent regression of the diseases or their clinical symptoms.

A “subject” includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like).

An “effective amount” as defined herein in relation to the treatment of a MDD or a condition that shares at least one of the two major defining symptoms of MDD, is an amount that will decrease, reduce, inhibit, or otherwise abrogate the MDD or a condition that shares at least one of the two major defining symptoms of MDD in the subject. An effective amount as used herein also includes an amount sufficient to delay the development of a symptom of a MDD or a condition that shares at least one of the two major defining symptoms of MDD, alter the course of MDD or a condition that shares at least one of the two major defining symptoms of MDD (for example but not limited to, slow the progression of a symptom of MDD and a condition that shares at least one of the two major defining symptoms of MDD, or reverse a symptom of MDD or a condition that shares at least one of the two major defining symptoms of MDD. The “effective amount” will vary depending on the whether the subject is suffering from MDD or a condition that shares at least one of the two major defining symptoms of MDD and its severity, as well as the age, weight, etc., of the subject to be treated. Additionally, the dosage can vary depending upon the dosage form employed and the route of administration utilized.

In some embodiments of a method for treating a subject suffering from MDD or a subject suffering from a condition that shares at least one of the two major defining symptoms of MDD, the method comprising administering an effective amount of a PARP1 inhibitor, the PARP1 inhibitor comprises a small molecule. In some embodiments, a small molecule PARP1 inhibitor comprises one or more of 3-aminobenzamide, 5-aminoisoquinolinone, Niraparib, Iniparib, Talazoparib, Olaparib, Rucaparib, and Veliparib. In certain embodiments, a small molecule PARP1 inhibitor comprises 3-aminobenzamide. In certain embodiments, a small molecule PARP1 inhibitor comprises 5-aminoisoquinolinone. In some embodiments, the PARP1 inhibitor comprises the inhibitor comprises PARP1 gene silencing siRNA.

It should be understood that the PARP1 inhibitors can include pharmaceutically acceptable salts, solvates, stereoisomers, and optical isomers thereof. It will further be understood that the compounds of PARP1 inhibitors, can include prodrugs of such compounds.

“Pharmaceutically acceptable salt” of a compound means a salt that is pharmaceutically acceptable and that possesses the desired pharmacological activity of the parent compound. As used herein, “pharmaceutically acceptable salt” refers to derivative of the compounds of the PARP-1 inhibitors, wherein such compounds are modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

It should be understood that all references to PARP1 inhibitors or pharmaceutically acceptable salts thereof, include solvent addition forms (solvates) or crystal forms (polymorphs) as defined herein.

The terms “crystal polymorphs” or “polymorphs” or “crystal forms” means crystal structures in which a compound (or salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Additionally, PARP1 inhibitors, for example, the salts of PARP1 inhibitors, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvates” means solvent addition forms that contain either stoichiometric or non stoichiometric amounts of solvent. Some compounds or salts have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

It should be understood that all references to PARP1 inhibitors can be prepared as prodrugs, for example pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.) Thus, PARP1 inhibitors can be delivered in prodrug form. Thus, the present invention is intended to cover prodrugs of PARP1 inhibitors, methods of delivering the same and compositions containing the same. “Prodrugs” are intended to include any covalently bonded carriers that release an active parent drug of the present invention in vivo when such prodrug is administered to a subject.

Exemplary modes of administration of the compounds of the combination therapy (e.g., a PARP1 inhibitor include, but are not limited to, injection, infusion, inhalation (e.g., intranasal or intratracheal), ingestion, rectal, and topical (including buccal and sublingual) administration. Local administration can be used if, for example, extensive side effects or toxicity is associated with the compounds of the combination therapy, and to, for example, permit a high localized concentration of the compounds to the infection site. Administration to deliver compounds of the combination therapy systemically or to a desired surface or target can include, but is not limited to, injection, infusion, instillation, and inhalation administration. Injection includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

In some embodiments of a method for treating a subject suffering from MDD or a subject suffering from a condition that shares at least one of the two major defining symptoms of MDD, the method comprising administering an effective amount of a PARP1 inhibitor, the method further comprises administration of an effective amount of a serotonergic and/or noradrenergic agent. In some embodiments, a serotonergic and/or noradrenergic agent comprises a noradrenergic-dopamine reuptake inhibitor. In some embodiments, serotonergic and/or noradrenergic agent comprises a serotonin reuptake inhibitor. In some embodiments, embodiments, the serotonin reuptake inhibitor comprises fluoxetine. In certain embodiments, the PARP1 inhibitor comprises 3-aminobenzamide and the serotonin reuptake inhibitor comprises fluoxetine. Again, it should be recognized that administration of an effective amount of a PARP1 inhibitor in combination with an effective amount of a serotonergic and/or noradrenergic agent can be used to treat a subject suffering from dysthymia, post-traumatic stress disorder, general anxiety disorder, and schizophrenia.

The serotonergic and/or noradrenergic agent can be administered by the same route or by different routes as the PARP1 inhibitor. For example, a PARP1 inhibitor of the combination treatment selected may be administered by intravenous, subcutaneous, or intraperitoneal injection, while the serotonergic and/or noradrenergic agent of the combination may be administered orally. Alternatively, for example, all therapeutic agents may be administered orally or all therapeutic agents may be administered by intravenous injection. The compounds in the particular combination therapy being used to treat a subject suffering from MDD or a subject suffering from a condition that shares at least one of the two major defining symptoms of MDD can determine the mode of administration to be used.

In some embodiments, the presently-disclosed subject matter includes a pharmaceutical composition comprising a therapeutically effective amount of a PARP1 inhibitor, a therapeutically effective amount of a serotonergic and/or noradrenergic agent, and a pharmaceutically-acceptable excipient.

In some embodiments of a pharmaceutical composition, a PARP1 inhibitor comprises a small molecule. In some embodiments, a small molecule PARP1 inhibitor comprises one or more of 3-aminobenzamide, 5-aminoisoquinolinone, Niraparib, Iniparib, Talazoparib, Olaparib, Rucaparib, and Veliparib. In certain embodiments, a small molecule PARP1 inhibitor comprises 3-aminobenzamide. In certain embodiments, a small molecule PARP1 inhibitor comprises 5-aminoisoquinolinone. In some embodiments, the PARP1 inhibitor comprises the inhibitor comprises PARP1 gene silencing siRNA

In some embodiments of a pharmaceutical composition, the serotonergic and/or noradrenergic agent comprises a noradrenergic-dopamine reuptake inhibitor. In some embodiments of a pharmaceutical composition, serotonergic and/or noradrenergic agent comprises a serotonin reuptake inhibitor. In some embodiments of a pharmaceutical composition, embodiments, the serotonin reuptake inhibitor is fluoxetine. In certain embodiments a pharmaceutical composition, the PARP1 inhibitor is 3-aminobenzamide and the serotonin reuptake inhibitor is fluoxetine, and a pharmaceutically-acceptable excipient.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Thus, the term “pharmaceutical excipient” is used herein to describe any ingredient other than the PARP1 inhibitor and the serotonergic and/or noradrenergic agent. Examples of pharmaceutical excipients include one or more substances which may act as diluents, flavoring agents, solubilizers, lubricants, suspending agents, binders, preservatives, wetting agents, tablet disintegrating agents, or an encapsulating material. The choice of excipient will to a large extent depend on factors such as the particular mode of administration, the effect of the excipient on solubility and stability, and the nature of the dosage form, as is understood by those of skill in the art. A “pharmaceutical excipient” includes both one and more than one such excipient.

The pharmaceutical composition comprising a therapeutically effective amount of a PARP1 inhibitor, a therapeutically effective amount of a serotonergic and/or noradrenergic agent, and at least one pharmaceutical excipient described herein can be formulated with a pharmaceutically acceptable carrier for administration to a subject. As such, the pharmaceutical compositions can be administered orally as a solid or as a liquid, or can be administered intramuscularly or intravenously as a solution, suspension, or emulsion. Alternatively, the pharmaceutical compositions can be administered by inhalation, intravenously, or intramuscularly as a liposomal suspension. In some embodiments, the pharmaceutical composition is formulated for oral administration. In other embodiments, the pharmaceutical composition is formulated for intravenous administration. As such, examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquids such as suspensions (e.g., aqueous or non-aqueous liquid suspensions, oil-in-water emulsions, or water-in-oil liquid emulsions), solutions, and elixirs; and sterile solids (e.g., crystalline or amorphous solids) that can be reconstituted to provide liquid dosage forms.

### EXAMPLES

The following examples are given by way of illustration and are in no way intended to limit the scope of the presently-disclosed subject matter.

### Example 1

### Material and Methods

Laboratory Animals: The use of animals for this study was approved by the University Committee on Animal Care at East Tennessee State University. All rats were ordered from Envigo, Inc. (Indianapolis, Ind., USA). Male Sprague-Dawley rats (225-250 g upon arrival) were used as subjects in the Porsolt swim test and were socially housed in groups of 2-3 per cage. Rats used as ‘intruders’ in the social defeat paradigm were individually housed and provided enrichment per the NIH Guidelines for the Care and Use of Animals. Intruder rats were male Sprague-Dawley rats (225-250 g). In addition, a total of 14 female Sprague-Dawley rats weighing 175-199 g upon arrival were obtained for the social defeat paradigm and these rats were socially housed for 6 days in the animal colony prior to fallopian tube ligature, performed as previously described. (Szebeni et al., 2016). Sixteen male Long-Evans hooded rats weighing 250-275 g upon arrival were used as ‘residents’ in the social defeat paradigm. A climate-controlled vivarium was utilized and animals were kept on a 12 h on/12 off light/dark cycle.

Social Defeat Stress (SDS): SDS was induced as described previously (Covington and Miczek, 2001; Szebeni et al., 2016). Briefly, Long-Evans hooded rats (‘residents’) were each mated with a female (ligated) rat for a 7-day period. On the eighth day and after removal of the female, an ‘intruder’ rat was placed into the cage for a 5 min period and dominance was established by the resident. Defeat was produced between 0900 and 1000 h daily for 10 consecutive days. Control rats not exposed to defeat were handled each day during this same time period.

Chronic unpredictable stress (CUS): CUS was performed after SDS on the same day, but at random times either during the day or evening as previously described (Bondi et al., 2008; Szebeni et al., 2016). Different stressors were randomly arranged and occurred at random times during the light or dark cycle of each day for 10 consecutive days. All rats were exposed twice to each of 5 different stressors, which included a 30-min restraint, a 1-h shaking/crowding, 10-min cold water (18° C.) swim, a 15-min warm water swim (25° C.) and a 24-h tipped cage. For restraint, rats were placed in a restraining device made of Plexiglas restricting movement but allowing free respiration and air circulation. In the shaking-crowding procedure, six rats were placed in a cardboard box atop a lab shaker set to produce 220 back-and-forth movements (approximately 2-in sideways deflection) per min. Both warm and cold swims were accomplished by placing the rat in a cylindrical tank (60 cm height×30 cm diameter) filled with water at a depth of 30 cm. For the tipped cage, the animal's home cage was tipped to one side by attaching a metal spring to one side of the cage to the cage rack for a 24 h period. Control rats were not exposed to the stressors, but were handled each day at the same time period.

Sucrose Preference: Sucrose preference was performed during the final three days of induction of social defeat stress (days 8-10), using a procedure based on that of D'Aquila et al. (1997). Animals were given two bottles on their cages between the 1900 and 2100 h on each day that it was performed (the first 2 h of the dark cycle) with one bottle containing tap water and the other containing 0.8% sucrose. Amounts of sucrose consumed were calculated as percentages of the total amount of fluid consumed during the 2 h period on each of the 3 days of testing. The position of the sucrose bottle (left or right) was alternated equally between groups and over days. The preference of sucrose over water was used as a measure of animal's sensitivity to reward and expressed as a percent.

Social interaction test: The social interaction test was performed 24 h after the last social defeat stress on day 11 of behavioral testing. The interaction test was conducted exactly as previously described (Brown et al., 2011). Animals were placed into a locomotor arena that was divided in half by a removable metal wire divider. The intruder was first placed into the area on one side of the divider and allowed to habituate for 5 min. After this period, a resident rat was placed on the other side of the divider. The amount of time spent in a defined interaction zone close to the metal divider was measured using ANYmaze video tracking (Stoelting Co., Wood Dale Ill., USA).

Porsolt swim test: Different groups of animals were tested in the Porsolt swim test. Rats were treated using the same drug treatment regimen as with the SDS/CUS paradigm, except as noted in the combined drug treatments as noted in the Results. On the eighth day of drug treatment, all animals began behavioral testing in the Porsolt swim test, also known as the forced swim stress test. All animals were tested in black cylinders measuring 36 cm in diameter, and these cylinders were filled with water of 23-25° C. following procedures as reviewed previously (Bogdanova et al., 2013), consistent with the original procedure of Porsolt and coworkers (Porsolt et al., 1978). All animals were given a pre-swim exposure test on the first day of testing, 24 h before the swim test session the following day. On the first day of testing, animals were exposed to the water for 15 min, and on the second day were given a 5 min trial. The two dependent measures used for forced swim stress was the latency to first immobility episode (immobility lasting >5 s) and the total immobility time over the 5 min period, both recorded on the second day of testing. All movements of the animal were recorded by behavioral scanning software (ANY-maze, Stoelting Co., Wood Dale, Ill., USA).

Drug administration: All drugs used in the study, 3-aminobenzamide (Product no. A0788; 3-AB), 5-AIQ hydrochloride (Product no. A7479), and fluoxetine (Product no. F132), were obtained from Sigma-Aldrich, Inc. (St. Louis, Mo., USA).

Statistical analysis: A Grubb's test was used to remove statistical outliers from each dataset prior to analyses. Statistical analyses were otherwise performed as indicated using IBM SPSS Statistics (version 23.0) and data were graphed using GraphPad Prism (version 5.0b, GraphPad Software, La Jolla, Calif.). An independent sample t-test was used to analyze data generated when only two groups were analyzed. An analysis of variance (ANOVA) was used to test multiple group comparisons. For post hoc statistical comparisons, a Bonferroni-correction was applied (as noted) to limit Type I error in multiple post-hoc comparisons. For the combined drug treatment experiment, ANOVA was followed by a Dunnett's Multiple Comparison test that focused comparisons of drug treatment groups to the vehicle control group. All data are expressed as mean±standard error of the mean.

### Example 2

### Results and Discussion

The present data demonstrates the antidepressant-like behavioral effects of PARP inhibitors in two different animal models used to typically to screen drugs for antidepressant activity in humans. Given that PARP inhibitors have no known direct effects on brain norepinephrine or serotonin, the findings here strongly implicate PARP inhibitors as an entirely novel type of antidepressant. Results here also suggest that PARP inhibitors could be used as an adjuvant to existing antidepressant treatments.

Drugs that inhibit PARP were known to have therapeutic potential in a number of different conditions. For example, the anti-cancer properties of PARP inhibitors are well known. Since cancer cells exploit PARP1 to protect themselves from death secondary to DNA lesions, PARP1 inhibitors facilitate the anti-cancer effects of DNA damaging anti-cancer drugs (e.g. cisplatin) and radiation therapy. Numerous PARP1 inhibitors are in clinical trials for cancer and one is currently marketed (olaparib, Astra-Zeneca, Inc.). With regards to the ability of PARP1 to facilitate NF-κB activation, PARP inhibitors have been recently demonstrated to have anti-inflammatory and neuroprotective actions in a number of different conditions associated with inflammation, including chronic asthma, myocardial infarction, stress-evoked immunocompromise, traumatic brain injury, and cerebral ischemia. However, this is the first study to demonstrate the ability of PARP inhibitors to counteract the deleterious effects of psychological stress on rodent behaviors and to produce antidepressant-like activity. The present data is based on the inventors' novel discovery that a type of brain cell (oligodendrocytes) demonstrates shortened telomeres in MDD brain donors, as compared to psychiatrically normal control brain donors. This was the first demonstration of shortened telomeres in brain cells in MDD. A follow up study also demonstrated shortened telomeres in hippocampal tissues from MDD brain donors. The inventors hypothesized that the shortened telomeres in the brain of MDD subjects may be a result of an elevated level of oxidative damage to these telomeres in MDD.

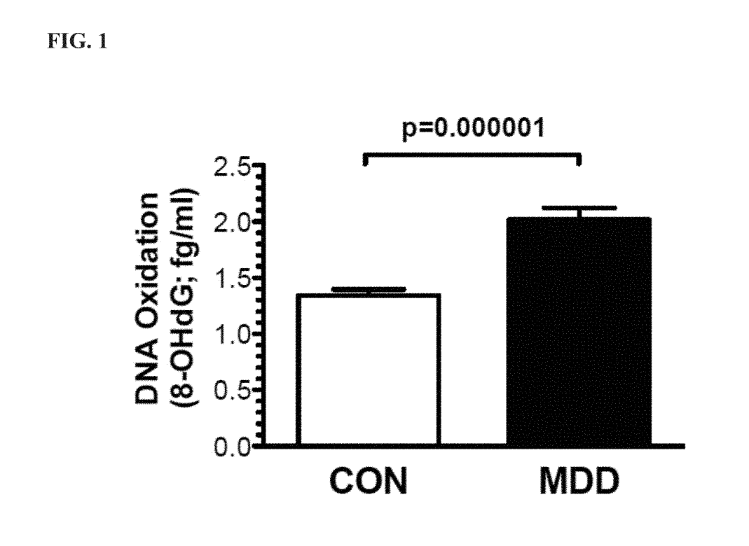

To examine this possibility, the inventors measured DNA oxidation in the area of the brain where they observed reduced telomere lengths in MDD using a commercially available ELISA kit. FIG. 1 shows that levels of DNA oxidation measured in DNA isolated from white matter obtained from postmortem brains from MDD and psychiatrically normal control donors. A significantly higher level of DNA oxidation was observed in MDD as compared to controls, supporting the notion that DNA oxidation may be the cause of shortened telomeres in brain cells in MDD.

When DNA is oxidized, there are proteins in the cell nucleus that are activated to correct the DNA damage. These proteins are referred to as base excision repair (BER) enzymes. One of these enzymes is PARP1, one of three PARP enzymes in the body. PARP1 is highly expressed in the brain. PARP1 is a sensor protein that detects DNA strand breaks that occur as a result of oxidized DNA along the DNA strand. Once it detects the strand break, its enzyme activity is activated and it poly(ADP-ribosyl)ates itself and other proteins. This ADPribosylation recruits other repair enzymes to the site to cut out the specific oxidized nucleotide from the DNA and insert a new nucleotide in its place.

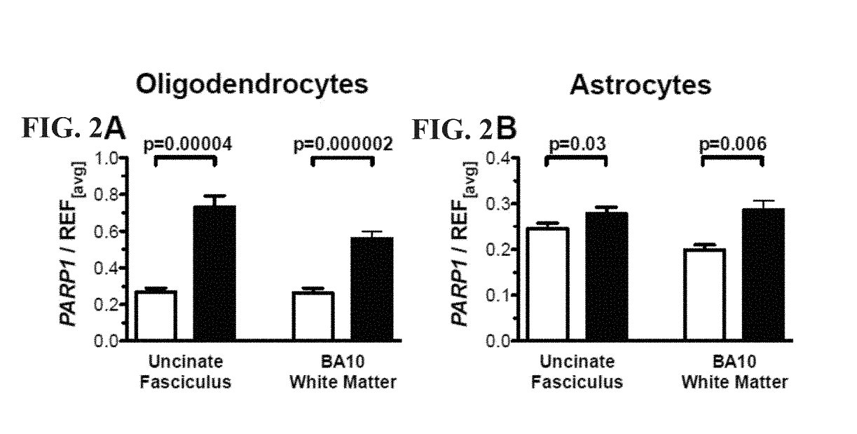

Gene expression of PARP1 is induced by conditions of oxidative stress, presumably as a compensatory mechanism to enhance base excision repair. Hence, the inventors postulated PARP1 gene expression may be altered in MDD. The inventors measured PARP1 gene expression in white matter cells from postmortem brain tissues of MDD and psychiatrically normal control subjects. The inventors observed a robust elevation of PARP1 gene expression in oligodendrocytes, and also in astrocytes, from two white matter regions in postmortem brains from MDD subjects (FIG. 2). Besides confirming the inventors' hypothesis that PARP1 would be affected by DNA oxidation in MDD, elevated PARP gene expression has other important implications particularly considering cellular consequences of elevated PARP activity.

Based on the data demonstrating increased PARP1 expression in MDD, the inventors hypothesized that the elevated PARP1 activity (as a result of elevated PARP1 gene expression) may induce an inflammatory cascade via NFκB activation and simultaneously deplete NAD+ and ATP. The inventors hypothesized that both of these actions would be expected to contribute brain cell dysfunction in susceptible brain regions, and ultimately could result in the development of major depression. To test the hypothesis that that PARP inhibition itself will produce an antidepressant effect, the inventors tested whether PARP inhibition will have antidepressant activity using two different rodent models involving psychological stress: 1) the Porsolt swim test, commonly used to identify antidepressant/anti-anxiety drugs (O'Leary O F, Cryan J F (2013) Towards translational rodent models of depression. Cell Tissue Res 354:141-153, herein incorporated by reference in its entirety); and 2) a combined repeated social defeat and repeated unpredictable stress model, both models of which are used to identify antidepressant effects (O'Leary and Cryan, 2013) and/or used to explore the biological effects of psychological stress in relation to posttraumatic stress disorder (Whitaker A M, Gilpin N W, Edwards S (2014) Animal models of post-traumatic stress disorder and recent neurobiological insights. Behav Pharmacol 25:398-409; Borghans B, Homberg J R (2015) Animal models for posttraumatic stress disorder: An overview of what is used in research. World J psychiatry 5:387-396, both herein incorporated by reference in their entireties). Two structurally different PARP inhibitors were tested, 3-AB and 5-aminoisoquinolinone (5-AIQ). Both of these drugs have been shown to be antagonists of PARP and both drugs have also been demonstrated to produce neuroprotective and/or anti-inflammatory actions in other disease models.

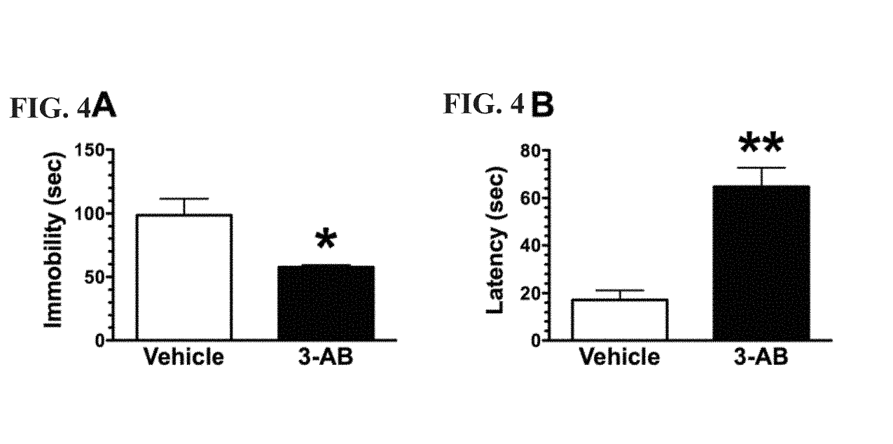

An initial preliminary experiment was conducted to examine the effects of 3-AB in the Porsolt swim test. Two groups of rats received either saline vehicle or 3-AB (40 mg/kg) i.p. daily for 10 days prior to swim testing. On the 10th day of treatment and 2 h after drug or vehicle injections, rats treated with 3-AB demonstrated a significantly decreased time spent immobile as compared to saline-treated controls on day 2 of the swim test (t=2.36, p<0.05; FIG. 4A). Additionally, 3-AB treated rats demonstrated a significant increase in the latency to immobility (t=5.56, p<0.001; FIG. 4B).

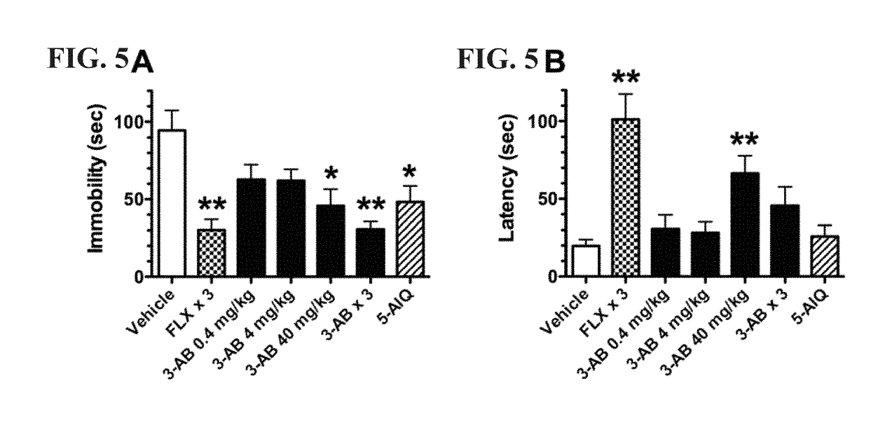

Based on these data, a more extensive experiment was conducted to examine the effect of PARP inhibitors in the Porsolt swim test. Three doses of 3-AB (0.4 mg/kg, 4 mg/kg and 40 mg/kg) were selected for study that were in the approximate range of doses shown to be effective in other disease models. In addition, a second PARP inhibitor, 5-AIQ, was tested at a dose of 0.3 mg/kg, a dose previously shown to have protective properties in a rat model of myocardial infarction. These treatments, and an additional group of rats treated with saline vehicle, were administered once daily for 10 days prior to behavioral testing. Two additional treatment groups were analyzed, including fluoxetine (10 mg/kg i.p.), and 3-AB (40 mg/kg i.p.; denoted 3-AB×3), both groups of which received injections 23.5, 5, and 1 h before behavioral testing. A one-way ANOVA of immobility time in the swim test revealed a significant main effect of treatment group (F=5.55, p<0.001). A posthoc Bonferroni comparison of the treatment groups of 5-AIQ, 3-AB 40/mg/kg (for 10 days), 3-AB×3, and fluoxetine were equivalent with respect to immobility times, and rats in these groups spent significantly less time immobile than rats in the vehicle group and in the rats treated with the two lower doses of 3-AB (0.4 and 4 mg/kg; FIG. 5A). For latency to immobility, one-way ANOVA revealed a significant main effect of group (F=9.08, p<0.001; FIG. 5B). Post-hoc analysis revealed that latencies of the fluoxetine group and rats treated with 40 mg/kg 3-AB for 10 days were equivalent and significantly greater than all vehicle-treated control rats. The two lower dose 3-AB groups, the 3-AB×3, the 5-AIQ treated group, and the vehicle control group did not significantly differ from one another. The statistical results of all group comparisons are shown in Tables 1 and 2, below.

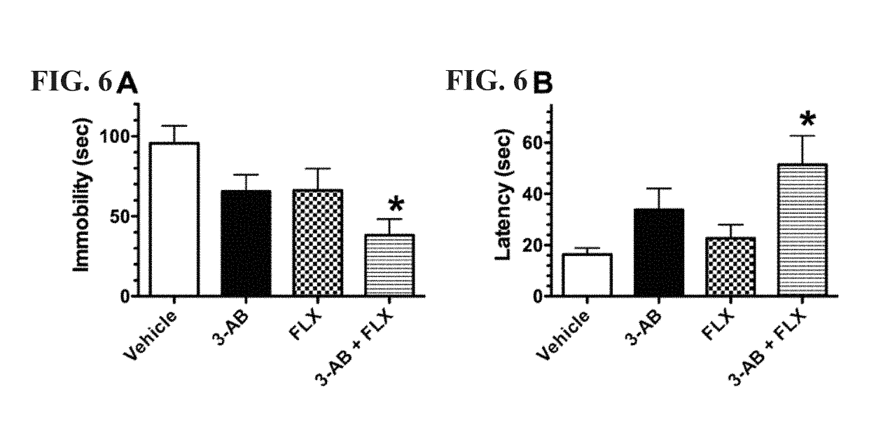

A third experiment was performed to determine whether 3-AB would increase the antidepressant activity of fluoxetine, again using the Porsolt swim test. Rats were treated with a dose of 3-AB (4 mg/kg; administered 3 times over 24 h) that was not observed in previous experiments to produce a significant effect on immobility time or latency to immobility (see FIGS. 5A and 5B). A dose of fluoxetine (2.5 mg/kg; administered 3 times over 24 h) was chosen that was expected to produce a less than maximal antidepressant response in the swim test. Both drugs were also administered together at the same doses and treatment schedule, as was saline vehicle. Analysis of data from this experiment revealed a significant group main effect on both immobility time (F=4.32, p=0.01; FIG. 6A) and latency to immobility (F=5.20, p=0.006; FIG. 6B). A Dunnett's test was used to compare each drug treatment group to the vehicle-treated group. Both 3-AB and fluoxetine alone did not significantly affect either immobility time or latency, while the combined treatment significantly reduced immobility (p<0.01) and significantly increased latency to immobility (p<0.01).

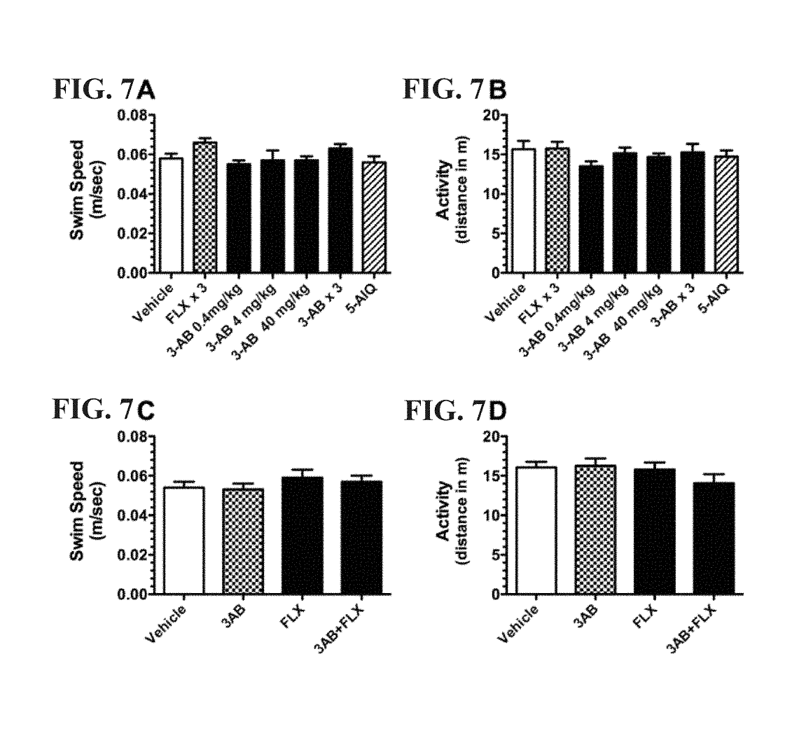

Drugs that increase locomotor activity can produce false positives in the Porsolt swim test. To consider the possibility that PARP inhibitors stimulate locomotor activity, two measures of activity were assessed for all rats of the second and third Porsolt swim experiments. Swim speed was assessed during the Porsolt swim procedure and locomotor activity was measured in an open field 24 h after the second day of the Porsolt swim test. Locomotor activity was assessed on the 24 h after the second swim (test day), at the same time after drug or vehicle injections as was performed for the swim test. There were no significant group differences in swim speed during the Porsolt swim test (F=1.57, p=0.170; FIG. 7A) or in locomotor activity tested the following day (F=0.956, p=0.463; FIG. 7B) in the second experiment (corresponding to FIG. 5A-B). Likewise, no significant group differences in swim speed (F=0.487, p=0.69; FIG. 7C) or locomotor activity (F=1.03, p=0.37; FIG. 7D) were observed in the third experiment (corresponding to FIG. 6A-B).

Experiments were next performed to determine whether the PARP inhibitor 3-AB would block the behavioral effects of repeated psychological stress. The rodent model of repeated stress in this study is rather unique, in that two stressors were administered as we have previously reported (Szebeni et al. Elevated DNA oxidation and DNA repair enzyme expression in brain white matter in major depressive disorder. Int J Neuropsychopharmacol, 20:363-373, herein incorporated by reference in its entirety). The rationale for using this double stress model was to reduce the likelihood of stress resilience. Others have constructed a theory of a “neurobiology of resilience” that occurs in both rodents and humans, wherein the rodent may be more well-adapted to develop resilience to stressors evolutionarily. Since SDS is typically performed at the same time each day, the rodent can predict over time when the stressor will occur, possibly enhancing resilience. Humans rarely experience stressors at the same time each day, a fact that weakens the construct validity of SDS. Thus, we used that the combination of a ‘mild” stressor of unpredictable nature (CUS) to the paradigm of SDS in attempts to improve construct validity, and presumably minimize the likelihood of rats to demonstrate resilience.

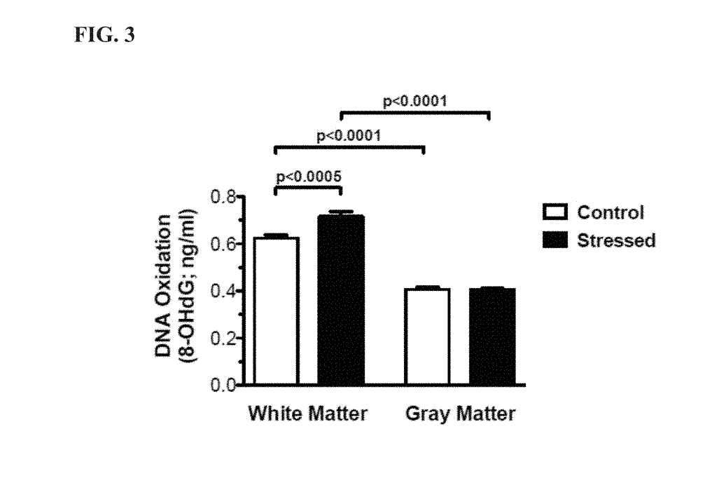

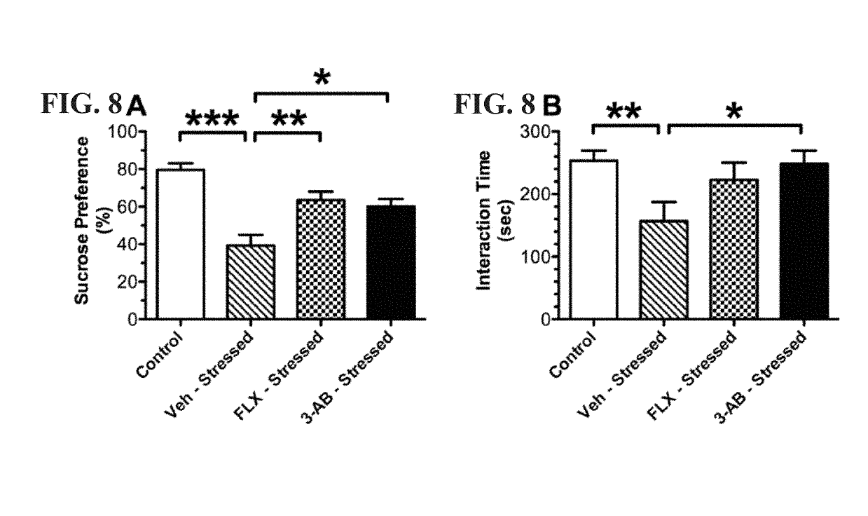

In this double stress model, rats were treated with vehicle, fluoxetine (10 mg/kg i.p. daily), or 3-AB (40 mg/kg i.p. daily) 2 h prior to the social defeat procedure each day for 10 days. These rats were also exposed daily to an unpredictable stressor. Rats receiving vehicle but no exposure to the two daily stressors served as a control group. DNA oxidation was significantly elevated in prefrontal cortical white matter after the 10 days of stress, as compared to rats that were housed and handled similarly but were not subjected to stressors (FIG. 3). No change was observed in cortical gray matter in the same rats. Analysis of variance revealed a significant main effect of group (F=12.91, p=2.7×10⁻⁵; FIG. 8A) on sucrose preference. Post-hoc analysis showed that vehicle-treated stressed rats had a robust reduction in sucrose preference relative to non-stressed control rats (p=1.1×10⁻⁵). Sucrose preference was significantly higher in stressed rats treated with 3-AB (p=0.024) or fluoxetine (p=0.005) as compared to stressed rats treated with vehicle, while 3-AB and fluoxetine groups did not significantly differ. There was a also a significant group main effect on time spent in the interaction zone (F=3.23, p=0.03; FIG. 8B). Vehicle-treated rats exposed to the stressors had a robust reduction of time in the interaction zone compared to control rats (p=0.008). Rats treated with 3-AB and exposed to stressors exhibited significantly greater interaction times compared to vehicle-treated rats exposed to stressors (p=0.014). Interaction times of the fluoxetine-treated rats appeared to be greater than that of vehicle-treated rats exposed to stress, although this difference did not reach statistical significance (p=0.073). The statistical results of all group comparisons of sucrose preference and interaction times are shown in Tables 3 and 4, below.

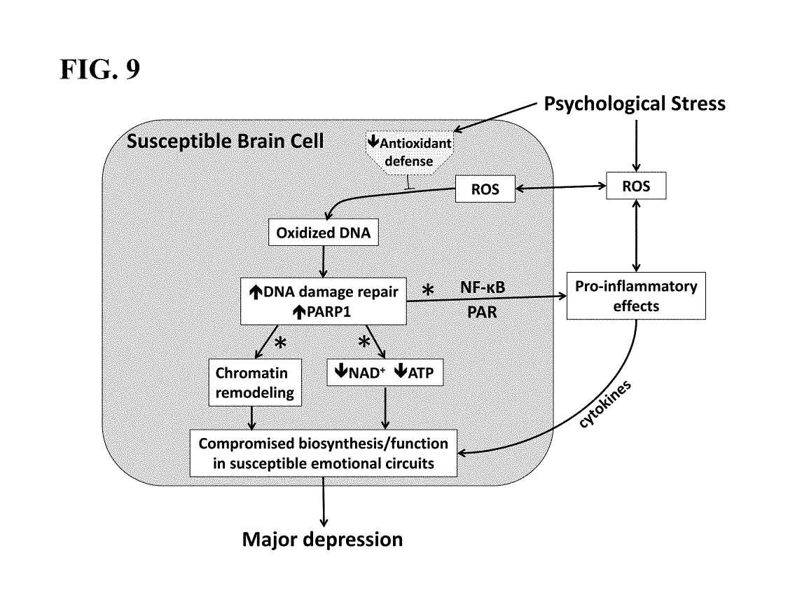

Therefore, as demonstrated by the data presented herein, PARP inhibitors demonstrate antidepressant-like behavioral effects in two different animal models used typically to screen drugs for antidepressant activity in humans. Two structurally different PARP inhibitors, 3-AB and 5-AIQ, demonstrated antidepressant-like activity in the Porsolt swim test. Importantly, both 3-AB and 5-AIQ produced their antidepressant-like responses in the swim test at doses that did not significantly affect locomotor activity or swim speed, suggesting that reduced immobility produced by these drugs was not secondary to a stimulant effect of the compounds. Further, 3-AB protected rats from the development of anhedonia and deficits in social interaction following repeated exposure to psychological stressors. Given that PARP inhibitors have no known direct effects on brain norepinephrine or serotonin, the findings here strongly implicate PARP inhibitors as an entirely novel type of antidepressant. FIG. 9 shows the two major pathways of brain cell damage that are predicted to be blocked by the administration of PARP inhibitors during psychological stress. It is noted that several psychiatric disorders are known to be precipitated by psychological stress. PARP inhibitor administration would be expected to be effective in any of these disorders, which include dysthymia, post-traumatic stress disorder, general anxiety disorder, and schizophrenia. Further, the combination of 3-AB plus fluoxetine produce antidepressant-like effects in the swim test at doses that did not produce a significant effect for either drug when administered alone. These results disclosed herein also suggest that PARP inhibitors can be used as an adjuvant or co-therapeutic to existing antidepressant treatments.

What is claimed:

1. A method for treating a subject suffering from major depressive disorder, the method comprising administering an effective amount of a poly(ADP)-ribose polymerase-1 (PARP1) inhibitor.

2. The method of claim 1, wherein the PARP1 inhibitor comprises a small molecule.

3. The method of claim 2, wherein the PARP1 inhibitor comprises 3-aminobenzamide.

4. The method of claim 1, wherein the PARP1 inhibitor comprises 5-aminoisoquinolinone.

5. The method of claim 2, further comprising administration of an effective amount of a serotonin reuptake inhibitor.

6. The method of claim 5, wherein the serotonin reuptake inhibitor comprises fluoxetine.

7. The method of claim 3, further comprising administering 3-aminobenzamide.

8. A method for treating a subject suffering from a condition characterized by either (1) depressed mood most of the day, nearly every day, for a period of at least two weeks or (2) markedly diminished pleasure or interest in all or almost all activities most of the day, nearly every day, for a period of at least two weeks, said characterizations defined and assessed in accordance with the Diagnostic and Statistical Manual of Mental Disorders, Fifth Edition, the method comprising administering an effective amount of a PARP1 inhibitor.

9. The method of claim 8, wherein the PARP1 inhibitor comprises a small molecule.

10. The method of claim 9, wherein the PARP1 inhibitor comprises 3-aminobenzamide.

11. The method of claim 8, wherein the PARP1 inhibitor comprises 5-aminoisoquinolinone.

12. The method of claim 9, further comprising administration of an effective amount of a serotonin reuptake inhibitor.

13. The method of claim 12, wherein the serotonin reuptake inhibitor comprises fluoxetine.

14. The method of claim 3, further comprising administering fluoxetine.

15. A pharmaceutical composition comprising:
a therapeutically effective amount of a PARP1 inhibitor;
a therapeutically effective amount of a serotonin reuptake inhibitor;
and a pharmaceutically-acceptable excipient.

16. The pharmaceutical composition of claim 15, wherein the PARP1 inhibitor comprises a small molecule.

17. The pharmaceutical composition of claim 16, wherein the PARP1 inhibitor comprises 3-aminobenzamide.

18. The pharmaceutical composition of claim 16, wherein the PARP1 inhibitor comprises 5-aminoisoquinolinone.

19. The pharmaceutical composition of claim 15, wherein the serotonin reuptake inhibitor comprises fluoxetine.
